(19) United States
(12) Patent Application Publication
(10) Pub. No.: US 2006/0159665 A1
(43) Pub. Date: Jul. 20, 2006

(54) Seed tear resistant scaffold

(75) Inventor: Bruno Giannetti, Bonn (DE)

Correspondence Address: MORGAN, LEWIS & BOCKIUS LLP, 1701 MARKET STREET, PHILADELPHIA, PA, 19103-2921, US

(73) Assignee: Verigen AG

(21) Appl. No.: 11/250,920

(22) Filed: Oct. 14, 2005

Related U.S. Application Data
(63) Continuation of application No. PCT/EP04/04192, filed on Apr. 21, 2004.

(51) Int. Cl. A61K 35/32; A61K 38/17; A61K 38/18; A61K 31/727

(52) U.S. Cl. 424/93.7; 424/422; 514/12; 514/56

(57) ABSTRACT

A seeded tear resistant scaffold comprising a biocompatible, tear resistant substrate, a biocompatible biodegradable material and optionally cells.

### BACKGROUND OF THE INVENTION

Collagen and gelatin have been applied as coatings, layers or as impregnations to textile grafts to avoid the need for preclotting the textile substrate prior to implantation. For example, U.S. Pat. Nos. 3,272,204, 4,842,575 and 5,197,977 disclose synthetic vascular grafts of this nature. Additionally, the '977 patent includes the use of active agents to enhance healing and graft acceptance once implanted in the body. The collagen source used in these patents is preferably from bovine skin or tendon dispersed in an aqueous solution that is applied to the synthetic textile graft by massaging or other pressure to cover the entire surface area and/or penetrate the porous structure.

U.S. Pat. No. 4,193,138 to Okita discloses a composite structure comprising a porous PTFE tube in which the pores of the tube are filled with a water-soluble polymer. The water-soluble polymer is used to form a hydrophilic layer which imparts an anti-thrombogenic characteristic to the ePTFE tube. Examples of such polymers are polyvinylalcohol, polyethylene oxides, nitrogen-containing polymers and avionic polymers such as polyacrylic acid and polymethacrylic acid. Additionally, hydroxy esters or carboxy esters of cellulose and polysaccarides are also disclosed. The '138 patent describes the diffusion of the water-soluble polymer into the pores of the tube and subsequent drying. The water-soluble polymer is then subjected to a crosslinking treatment to render it insoluble in water. Crosslinking treatment such as heat treatment, acetalization, esterification or ionizing radiation-induced crosslinking reactions are disclosed. The water-soluble materials disclosed in the '138 patent are synthetic in nature.

### SUMMARY OF THE INVENTION

In one embodiment, a seeded tear resistant scaffold of the present invention includes a biocompatible, tear resistant substrate, such as polytetrafluoroethylene, a semi-solid, solid, gel, and/or liquid biocompatible biodegradable material, such as collagen, and cells. The cells can be dispersed in and/or on a surface of (hereafter referred to as “on”) and/or adjacent to and/or throughout a biodegradable material and/or substrate. In one embodiment, a biocompatible, biodegradable material can be selected from generally extracellular matrix proteins, as will be further described hereinbelow.

### BRIEF DESCRIPTION OF THE FIGURE

FIG. 1 shows a portion of an e-PTFE substrate, a biodegradable material and cells.

FIG. 2 shows a portion of an e-PTFE substrate, a biodegradable material and cells formed into a surgical mesh or patch.

### DETAILED DESCRIPTION

A tear resistant scaffold of the present invention can contain two or more materials. The materials can include, but are not limited to, 1) a biocompatible substrate which provides support for the tear resistant scaffold and imparts tear resistance; and 2) a biodegradable, biocompatible material. A seeded tear resistant scaffold of the present invention includes a tear resistant scaffold and further includes cells.

Methods of manufacture of a tear resistant scaffold are described in more detail below. In one embodiment, a method of manufacture of a seeded tear resistant scaffold includes contacting one or more substrates with one or more biodegradable material and one or more cells. By “seed” or “seeding” or “seeded” is meant that cells are brought into contact with a support matrix and/or substrate, typically a biodegradable support matrix described in further detail below, and adhere (with or without an adhesive) on and/or in and/or adjacent to and/or throughout the support matrix and/or substrate for a period of time prior to transplantation.

A seeded tear resistant scaffold can then be used by implanting the tear resistant scaffold into a patient to repair a defect in a tissue type, including but not limited to cartilage, muscle, tendon, ligament, bone, and intervertebral disc tissue.

Each of the materials, methods of manufacture and use are described in more detail below.

A. The Materials of the Tear Resistant Scaffold

1. Substrate

A substrate portion of a tear resistant scaffold of the present invention can be constructed from one or more biocompatible materials. Examples of such materials include, but are not limited to, non-biodegradable materials such as polytetrafluoroethylene, perfluorinated polymers such as fluorinated ethylene propylene, polypropylene, polyethylene, polyethylene terapthalate, silicone, silicone rubber, polysufone, polyurethane, non-degradable polycarboxylate, non-degradable polycarbonate, non-degradable polyester, polyacrylic, polyhydroxymethacrylate, polymethylmethacrylate, polyamides such as polyesteramide, and copolymers, block copolymers and blends of the above materials. The above described materials can also be crosslinked or non-crosslinked.

Preferably, a substrate can be constructed of expanded polytetrafluoroethylene (ePTFE), including but not limted to ePTFE materials such as Gore-Tex® (available from W. L. Gore & Associates, Inc., 555 Papermill Road Newark, Del.) which is an extremely inert and biocompatible material with a history of medical implant use. U.S. Pat. Nos. 3,953,566 and 4,187,390, the disclosures of which are incorporated herein by reference, teach methods for producing ePTFE suitable for use in the present invention.

In another embodiment, a substrate includes a material that has pores. The pore size is dependent on the processing and stretching parameters used in preparation of the substrate. For purposes of this invention, the term “pores” will be used interchangeably with other terms such as interstices, voids and channels. In another embodiment, a substrate includes a material that has substantially no pores.

In another embodiment, a substrate surface can be chemically modified to impart greater hydrophilicity thereto. For example, this can be accomplished by glow discharge plasma treatment or other means whereby hydrophilic moieties are attached to or otherwise associated with the substrate surface. Such treatment enhances the ability of the substrate to imbibe the biocompatible dispersion/solution, as described below.

The substrate can be cut into any regular or irregular shape. In a preferred embodiment, the substrate can be cut to correspond to the shape of the defect. The substrate can be flat, round and/or cylindrical in shape. The shape of the substrate can also be molded to fit the shape of a particular tissue defect. If the substrate is a fibrous material, or has the characteristics of a fiber, the substrate can be woven into a desired shape. Alternatively, the substrate can be a non-woven material.

2. Biodegradable Materials

As described below, one or more biocompatible, biodegradable materials for use with the present invention can then be added to a substrate as a coating, layer and/or impregnation. As used herewith the term “biodegradable” means it will break down and/or be absorbed in the body. Suitable materials include, but are not limited to, extracellular matrix proteins which are known to be involved in cell-to-cell adhesion mechanisms. The materials can be natural or synthetic, and can be in a solid, semi-solid, gel or liquid form.

These materials can be one or more of the extracellular matrix proteins including, but not limited to, collagen (including e.g., collagen types I-V), gelatin, vitronectin, fibronectin, laminin, reconstituted basement membrane matrices, hyaluronic acid, hydrolyzable polyesters such as polylactic acid and polyglycolic acid, polyorthoesters, degradable polycarboxylates, degradable polycarbonates, degradable polycaprolactones, polyanhydrides, and copolymers, and biodegradable block copolymers and blends of the above materials. Biodegradable materials can be used either alone or in combination, and can be cross linked or non-cross linked. Other suitable biodegradable materials are described in U.S. patent application Ser. No. 10/121,249, the entire content of which is hereby incorporated by reference.

Types of commercial products which can be used in this invention include, but are not limited to Surgicel®, Surgicel® W1912 (Lot GG3DH), available from Ethicon Ltd., UK, ChondroCell® (a commercially available type II collagen matrix pad, Ed. Geistlich Sohne, Switzerland), and Chondro-Gide® (a commercially available type I collagen matrix pad, Ed. Geistlich Sohne, Switzerland), as well as a cross-linked or uncross-linked form of Permacol™ (Tissue Science Laboratories, UK). Other biodegradable materials similar to Permacol™, such as the Rapi-Seal Patch (Fusion Medical Technologies, Inc., Fremont, Calif.) and the Tissue Repair Patch (Glycar Vascular Inc., Dallas, Tex.), may also be used in the present invention. Additional materials which can be useful in the present invention are the Small Intestine Submucosa (“SIS”) materials, and in the present invention can include, but are not limited to, the Suspend Sling™ from Mentor Corporation (Santa Barbara, Calif.), Staple Strips™ from Glycar Vascular, Inc. (Dallas, Tex.), Surgical Fabrics from Boston Scientific (Natick, Mass.), SurgiSIS™ Sling and SurgiSIS™ Mesh from Cook Biotech, Inc. (West Lafayette, Ind.), SIS Hernia Repair Device from Sentron Medical, Inc. (Cincinnati, Ohio), and the Restore® Soft Tissue Implant from DePuy Orthopaedics.

Other collagen materials that can be used as a biodegradable material according to the present invention include, but are not limited to, FortaFlex™ (prepared from collagen type I) and GraftPatch® (prepared from cross-linked collagen) from Organogenesis, Inc. (Canton, Mass.). Additionally, Antema®, an equine collagen type I composition from Opicrin S.p.A. (Corlo, ITALY), is also useful in the present invention.

Other biodegradable materials suitable for use in the present invention include, but are not limited to, CollaTec membrane from Colla-Tec, Inc. (Plainsboro, N.J.), Collagraft from NeuColl (Campbell, Calif.), BioMend from Integra Life Sciences Corporation (Plainsboro, N.J.), and BioMend® Absorbable Collagen Membrane from Collagen Matrix, Inc. (Franklin Lakes, N.J.). Biosynthetic Surgical Mesh from Advanced UroSciences, Inc., Brennen Medical, Inc. (St. Paul, Minn.), which is prepared from porcine skin (essentially all collagen) and BIOBAR™ from Col-Bar, Ltd. (Ramat-Hasharon, Israel).

A particularly suitable biodegradable material will be solid, semi-solid or gel-like, characterized by being able to hold a stable form for a period of time to enable the growth of cells thereon, both before transplant and after transplant, and to provide a system similar to the natural environment of the cells to optimize cell growth and differentiation. Examples of suitable materials are disclosed in U.S. patent application Ser. No. 10/121,249, which is hereby incorporated by reference in its entirety.

3. Cells

As indicated above, a seeded tear resistant scaffold of the present invention can be a tear resistant scaffold that further includes cells. Suitable autologous or non-autologous cell types for use with the present invention include, but are not limited to nonepithelial cells including 1) fibroblasts, including but not limited to cells of the loose connective tissue such as ligaments and tendons, and the reticular tissue of bone marrow, as well as 2) the nucleus pulposus cell of the intervertebral disc, 3) cementoblasts/cemontocytes, and 4) odontoblasts/odontocytess, as well as other types of cells, including but not limited to synoviocytes. In other embodiments, suitable autologous or non-autologous cell types for use with the present invention include, but are not limited to muscle cells, soft tissue cells, bone cells including but not limited to osteocytes, tendon cells including but not limited to tenocytes, nerve cells, and cartilage cells including but not limited to chondrocytes.

In some embodiments of the invention, the methods may also include use of non-autologous and/or autologous stem cells from any source. A background and detailed description of autologous transplantation is found in U.S. Pat. No. 6,379,367, which is herein incorporated by reference in its entirety.

It is believed that the number of cells used to seed the tear resistant scaffold of the present invention does not limit the final tissue produced, however optimal seeding may increase the rate of generation. Optimal seeding amounts will depend on the specific culture conditions (described in more detail below). In one embodiment, the tear resistant scaffold can be seeded with from about 0.05 to about 5 times the physiological cell density of a native tissue type, e.g., native tendon, ligament and/or disc tissue. In another embodiment, the cell density can be less than about 1×10⁵ to 1×10⁸ cells per ml. or more, typically about 1×10⁶ cells per ml.

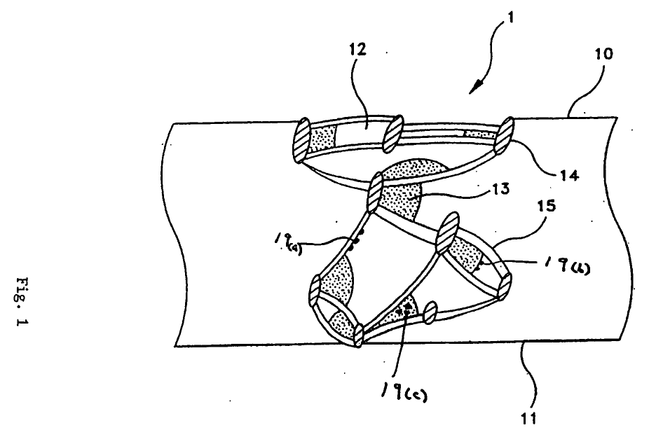

FIG. 1 shows a seeded tear resistant scaffold of the present invention. As shown in FIG. 1, a portion of an expanded PTFE substrate 1 having sides 10 and 11, nodes 14, tear resistant substrate 15, pores 12, also can include biocompatible, biodegradable material 13 and cells 19. Biodegradable material 13 at least partially fills some or all of the pores 12 of substrate 1. In FIG. 1, cells 19(a) are on and/or adjacent to and/or adhered to a surface of substrate 1, as well as on and/or adjacent to and/or adhered to a surface of biodegradable material 13, shown by cells 19(b), as well as in and/or throughout biodegradable material 13, shown by cells 19(c).

4. Other

A tear resistant scaffold and/or a seeded tear resistant scaffold of the present invention can also include various pharmacological actives including but not limited to antimicrobials, antivirals, antibiotics, growth factors, blood clotting modulators such as heparin and the like, as well as mixtures and composite layers thereof can be added to the biocompatible biodegradable material, prior to impregnation into the substrate.

A tear resistant scaffold and/or a seeded tear resistant scaffold of the present invention can also include growth factors such as autologous and non-autologous growth factors, including but not limited to transforming growth factor (TGF-β3), bone morphogenic protein (BMP-2), PTHrP, osteoprotegrin (OPG), Indian Hedgehog, RANKL, and insulin-like growth factor (IgF1), as described in U.S. patent application Ser. No. 10/254,124, the entire content of which is hereby incorporated by reference.

The present invention can also include a biocompatible glue in contact with a substrate and/or biodegradable material and/or cells. Such biocompatible glues or adhesives can include an organic fibrin glue (e.g., Tisseel®, fibrin based adhesive, Baxter, Austria or a fibrin glue prepared in the surgical theater using autologous blood samples). In one embodiment, cells of the present invention can be mixed with an appropriate glue before, during and/or after contact with a tear resistant scaffold of the present invention. Alternatively, an appropriate glue can be placed in a defect or layered on top of cells or as a layer below cells on or impregnated in a tear resistant scaffold of the present invention.

In one embodiment, the present invention includes cells and glue combined together in a mixture of glue and cells or one or more alternating layers of cells and glue on a tear resistant scaffold. It is contemplated that cells are autologous can be transplanted into a defect. Cells are mixed, either homogeneously or non-homogeneously, with a suitable glue before application of the cell/glue mixture to a tear resistant scaffold. Preferably, the glue and the cells are mixed immediately (that is, in the operating theater) before applying the glue and cells to the tear resistant scaffold and implantation of the combination of glue, cells and tear resistant scaffold to a defect. Alternatively cells and a glue are alternately applied in one or more layers to support a tear resistant scaffold. In one embodiment, a glue for use in the present invention is a bio-compatible glue, such as a fibrin glue, and more specifically either an autologous fibrin glue or a non-autologous fibrin glue. Preferably, an autologous fibrin glue is used.

B. A Method of Making the Tear Resistant Scaffold and Seeded Tear Resistant Scaffold

1. Tear Resistant Scaffold

In one embodiment, one or more of the above described biodegradable materials can be introduced to a substrate having pores or substantially no pores, preferably via aqueous dispersion or solution and precipitated out to form a solid, gel or semi-sold. Optionally, the biodegradable material can undergo crosslinking to form body fluid insoluble materials. The biodegradable material can be applied as a layer, coating and/or impregnation of the substrate. Alternately, a biocompatible, biodegradable material can be introduced to a porous substrate or a substrate having substantially no pores in a solid, semi-solid, gel and/or liquid form using fluid-pressure or other techniques such as pre-crosslinking.

In one embodiment, a biodegradable material as described herein above coats or layers on a portion of a porous substrate or a portion of a substrate that has substantially no pores. In another embodiment, rather than coat or layer a portion of the substrate, a biodegradable material can serve as a filler for the voids of a substrate having pores. More specifically, if a porous substrate is used, a biocompatible, biodegradable material preferably substantially fills at least a portion of the voids of the substrate material and can provide a cellular binding surface for tissue regrowth, as shown in FIG. 1.

In one embodiment, the process of preparing the substrate of the present invention includes using a force to cause a biodegradable dispersion of biocompatible material to penetrate into the voids of a substrate having voids, thereby contacting internodal voids, as shown in FIG. 1. This can be accomplished in a number of ways, such as by using pressure (e.g., vacuum) to cause migration of a biodegradable dispersion if the biodegradable material into the interstices of the substrate walls. The flow of the dispersion is believed to permit sufficient contact between the biocompatible, biodegradable materials and the voids. While impregnation time depends on the substrate pore size, graft length, impregnation pressure, biodegradable material concentration and other factors, generally it can be accomplished in a short period of time, for example from less than 1 minute to 10 minutes at a preferred temperature range of about 25 to 35° C. These parameters are not critical however, provided the voids are substantially filled with the biocompatible, biodegradable material.

A biocompatible, biodegradable material may be optionally subjected to crosslinking treatment such that it is solidified in place. For example, crosslinking by exposure to various crosslinking agents and methods such as formaldehyde vapor can then be preferably carried out. Subsequent to formation of the cross-linked collagen, the prosthesis can then be rinsed and prepared for sterilization by known methods. Vacuum drying or heat treatment to remove excess moisture and/or crosslinking agents can then be used. The entire process of contacting a substrate with a biodegradable dispersion/solution can be repeated several times, if necessary, to achieve the desired impregnation, coating and/or layering.

After a biodegradable material is contacted with a non-biodegradable substrate, a tear resistant scaffold can be formed. However, in order to form a seeded tear resistant scaffold, cell seeding must also take place, which is described below.

2. Cell Seeding

2a. Cell Cultivation

Initially, for autologous cells, a patient undergoes a procedure referred to as arthroscopy. This is a minimally invasive technique, usually performed in an outpatient setting. A small periscope-like device is inserted into the area of the defect to allow the surgeon to visualize the inside of patient's body and the area surrounding the defect. If the surgeon diagnoses a ligament, tendon or disk defect, the surgeon can perform a biopsy procedure to retrieve a tiny sample of healthy tissue. The healthy tissue biopsy preferably is of the same tissue type (i.e., tendon, ligament and/or nucleus pulposus cells of the intervertebral disc) that has the defect.

Next, the biopsy tissue can be sent to a processing facility. There, the cells from the biopsy can be nourished and grown in culture. The growth can take from several days to several weeks.

If stem cells are to be used, autologous stem cells can be obtained from the subjects' blood, bone marrow, stored umbilical cord blood, etc. The cells can be sent to a processing facility. There, the stem cells can be nourished and grown in culture. The growth can take from several days to several weeks.

A culture of non-autologous cells (e.g., cells from another person and/or fetal tissue) including but not limited to the cells described herein, can also be used. The method of culturing such cells is described in the following reference: Freshney (2000, Culture of Animal Cells: A Manual of Basic Techniques, 4th Edition, Wiley-Liss, New York, N.Y.), the entire content of which is hereby incorporated by reference.

2b. Cell Seeding

Following culturing, the cells are then ready for return to the patient via combination with the substrate and/or biodegradable material, as described below.

One or more cells, including but not limited to the cells described herein, can be obtained from culture and seeded within a biodegradable material dispersion (described above) either pre- or post- matrix formation, depending upon the particular matrix used and the method of matrix formation. Uniform seeding is preferable. As noted above, it is believed that the number of cells seeded does not limit the final tissue produced, however optimal seeding may increase the rate of generation. Optimal seeding amounts will depend on the specific culture conditions. In one embodiment, the matrix is seeded with from about 0.05 to about 5 times the physiological cell density of a native tissue type, i.e., tendon, ligament and/or disk tissue. In another embodiment, the cell density can be less than about 1×10⁵ to 1×10⁸ cells, or more, per ml., typically about 1×10⁶ cells per ml.

A dispersion of a biodegradable material, described above, can also contain one or more cells described herein, including but not limited to fibroblasts and nucleus pulposus cell of the intervertebral disc cells and combinations thereof. A dispersion containing cells can be contacted with the substrate to form a seeded tear resistant scaffold of the present invention having cells in and/or on and/or throughout and/or adjacent to the tear resistant scaffold.

Alternatively, the cells can be applied in, on and/or adjacent to and/or throughout one or more surfaces of a substrate material and/or a biodegradable material before, during or after the substrate has been contacted with a biodegradable material.

A suitable device and method for seeding a tear resistant scaffold of the present invention is described in pending U.S. patent application Ser. No. 10/047,571, the entire content of which is hereby incorporated by reference.

Once combined, the substrate material, biodegradable material and cells form a seeded tear resistant scaffold of the present invention.

3. Embodiments

In another embodiment, a tear resistant scaffold of the present invention can be seeded with multiple cell types and have different cell types on and/or in and/or throughout and/or adjacent to different portions of the scaffold. By way of example, one portion of the scaffold may include a first cell type (e.g., tendon cells) and another portion of the scaffold may include a second cell type (e.g., ligament cells). By way of further example, if the tear resistant scaffold is disc shaped, having two sides and an edge, a first side can include a first cell type (e.g., tendon cells) thereon and the second side can include a second cell type (e.g., ligament cells) thereon. Alternatively, each surface of a disc shaped tear resistant scaffold can include the same cell type in and/or on and/or throughout and/or adjacent to a surface.

In another embodiment, two or more substrates can be in contact with each other. In such an embodiment, a first substrate can be in contact with a second substrate either before, during or after either substrate is contacted with a biodegradable material to form a tear resistant scaffold or before, during or after either substrate is contacted with cells, as described above.

In another embodiment, two or more tear resistant scaffolds can be in contact with each other. In such an embodiment, the tear resistant scaffolds can be layered together. The layering can occur before or after the tear resistant scaffold has been seeded with one or more cells described herein.

Alternatively, two or more tear resistant scaffolds can be separated by an additional layer of biodegradable material and/or substrate material which can be sandwiched therebetween. Preferably such a layer includes one or more of the biodegradable and/or substrate materials described above. The layer separating tear resistant scaffolds can also optionally contain cells in, on and/or throughout and/or adjacent to the separating layer, in the manner described above. The cells present in each seeded tear resistant scaffold and/or layer of biodegradable material and/or layer of substrate material can be the same cell type or different cell type relative to adjacent layers of biodegradable material and/or substrate and/or tear resistant scaffold.

C. The Use of the Tear Resistant Scaffold

A tear resistant scaffold of the present invention and/or a seeded tear resistant scaffold of the present invention can be used to repair tissue defects. A repair can be effected in a variety of manners apparent to one of skill in the art in view of the teaching herein, including but not limited to the following.

By way of example, and not by limitation, the present invention provides a method for treating tendon tears by transplanting autologous tenocytes onto a tear resistant scaffold. One representative example of a tendon tear is rotator cuff tendonitis, caused by a partial tendon tear. The invention also includes methods for implantation of the tenocyte-seeded tear resistant scaffold into the site of transplantation.

The present invention also contemplates use of the methods taught in the invention to treat ligament defects. In one embodiment, autologous ligament cells are seeded on the tear resistant scaffold and the cell-seeded tear resistant scaffold can be implanted into the site of transplantation. The present invention also provides a method for implantation of the cell-seeded tear resistant scaffold into the site of transplantation.

The present invention also contemplates use of the methods taught in the invention to treat intervertebral disc defects. In one embodiment, autologous pulposus cells of the intervertebral disc are seeded on the tear resistant scaffold and the cell-seeded tear resistant scaffold can be implanted into the site of transplantation. The present invention also provides a method implantation of the cell-seeded tear resistant scaffold into the site of transplantation.

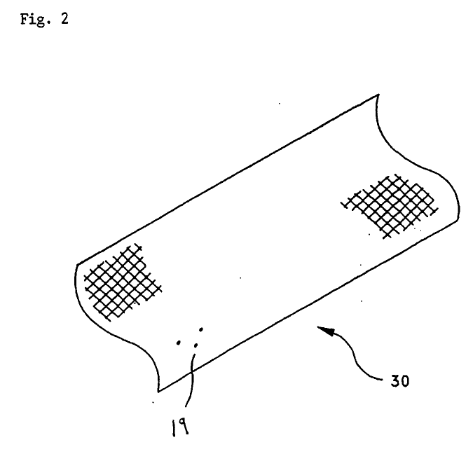

After one or more cells, biodegradable materials and substrates are combined in an appropriate manner, a seeded tear resistant scaffold can be implanted into the patient to repair the defect. A suitable seeded tear resistant scaffold is shown in FIG. 2. Specifically, FIG. 2 shows a seeded tear resistant scaffold of FIG. 1 formed into an implantable surgical mesh 30 having cells 19 disposed on a surface of mesh 30.

To accomplish a repair of a defect, an incision is typically made in the area of the defect to expose the defect. Damaged tissue is then typically removed, and the defect area is prepared to receive the tear resistant scaffold of the present invention. The tear resistant scaffold can then be surgically implanted into the patient to repair the defect. The cells that adhere to the tear resistant scaffold gradually regenerate new tissue that eventually grows to appear and function like the original tissue.

### EXAMPLES

### Example 1

Expanded PTFE starting materials are manufactured following the methods described in Example 1 of U.S. Pat. No. 5,032,445, issued to Scantlebury et al. which is incorporated herein by reference.

### Example 2

A biopsy can be taken from the tendon of flexor carpi radialis or calcaneus tendon, and washed in DMEM, then cleaned of adipose tissue. The tissue is minced and digested in 0.25% trypsin in serum-free DMEM for 1 hour at 37° C., followed by 5 h digestion in 1 mg/ml collagenase in serum-free Dulbecco's Modified Essential Medium (DMEM) at 37° C. The cell pellet is washed 2-3 times (centrifuged at 200 g for about 10 minutes), and resuspended in growth medium (DMEM containing 10% fetal calf serum, 50 ug/ml ascorbic acid, 70 micromole/liter gentamycin sulfate, 2.2 micromole/liter amphotericin. The tenocytes are counted to determine viability and then seeded. The culture is maintained in a humidified atmosphere of 5% CO₂, 95% air in a CO₂ incubator at 37 degrees Celsius and handled in a Class 100 laboratory. The medium is changed every 2 to 3 days. Other compositions of culture medium may be used for culturing the cells. The cells are then trypsinized using trypsin EDTA for 5 to 10 minutes and counted using Trypan Blue viability staining in a Buurker-Turk chamber. The cell count is adjusted to 7.5×10⁵ cells per milliliter.

A e-PTFE material is impregnated with type I/III collagen to form a tear resistant scaffold. The scaffold is cut to a suitable size to fit the bottom of the well in the NUNCLON™ Delta 6-well tissue culture tray and placed in the well under aseptic conditions (NUNC (InterMed) Roskilde, Denmark). A small amount of the cell culture medium containing serum is applied to the matrix to be absorbed into the matrix and to keep the matrix wet at the bottom of the well.

Approximately 10⁶ cells in 1 milliliter of culture medium are placed directly on top of the scaffold, dispersed over the surface of the scaffold. The tissue culture plate is then incubated in a CO₂ incubator at 37 degrees Celsius for 60 minutes. From 2 to 5 milliliters of tissue culture medium containing 5 to 7.5% serum is carefully added to the tissue culture well containing the cells. The pH is adjusted to about 7.4 to 7.5 if necessary. The plate is incubated for 3 to 7 days with a medium change at day 3.

At the end of the incubation period the medium is decanted and the cell-seeded tear resistant scaffold is washed. The tear resistant scaffold is then implanted, into the defect site. The defect is then permitted to heal on its own.

It will be appreciated by persons skilled in the art that numerous variations and modification may be made to the invention shown in the specific embodiments without departing from the spirit or scope of the invention as broadly described. The present embodiments and examples are, therefore, to be considered in all respects as illustrative and not restrictive.

Each and every reference cited herein is hereby incorporated by reference.

1. A seeded tear resistant scaffold comprising a biocompatible, tear resistant substrate, a biocompatible biodegradable material and optionally cells.

2. The scaffold according to claim 1, wherein the substrate comprises at least one of polytetrafluoroethylene, perfluorinated polymers, polypropylene, polyethylene, polyethylene terapthalate, silicone, silicone rubber, polysufone, polyurethane, non-degradable polycarboxylate, non-degradable polycarbonate, non-degradable polyester, polyacrylic, polyhydroxymethacrylate, polymethylmethacrylate, polyamides, copolymers and, block copolymers.

3. The scaffold according to claim 1, wherein the biocompatible biodegradable material is at least one of a semi-solid, a solid, a gel, and a liquid material.

4. The scaffold according to claim 1, wherein the biocompatible biodegradable material is selected from the group consisting of collagen, gelatin, vitronectin, fibronectin, laminin, reconstituted basement membrane matrices, hyaluronic acid, hydrolyzable polyesters, polyorthoesters, degradable polycarboxylates, degradable polycarbonates, degradable polycaprolactones, polyanhydrides, copolymers, and biodegradable block copolymers and combinations thereof.

5. The scaffold according to claim 1, wherein the cells are at least one of dispersed in a surface and dispersed on a surface of at least one of a biodegradable material and substrate.

6. The scaffold according to claim 1, wherein the biocompatible, biodegradable material is extracellular matrix proteins.

7. The scaffold according to claim 2, wherein the substrate is cross-linked.

8. The scaffold according to claim 2, wherein the substrate is an expanded polytetrafluoroethylene (ePTFE).

9. The scaffold according to claim 1, wherein the biocompatible tear resistant substrate is porous.

10. The scaffold according to claim 9, wherein the porous substrate comprises pores that allows cells to penetrate the porous substrate.

11. The scaffold according to claim 1, wherein a surface of the substrate is chemically modified.

12. The scaffold according to claim 1, wherein the substrate is formed in at least one of a regular and irregular shape.

13. The scaffold of claim 1, comprises at least one of a woven and non-woven fabric.

14. The scaffold according to claim 1, wherein the cells are at least one of autologous and non-autologous and are selected from the group consisting of fibroblasts, cells of the loose connective tissue cells of the reticular tissue of bone marrow, nucleus pulposus cell of the intervertebral disc, cementoblasts/cemontocytes, odontoblasts/odontocytes, synoviocytes, muscle cells, soft tissue cells, bone cells such as osteocytes, tendon cells nerve cells, cartilage cells, and stem cells from any source.

15. The scaffold according to claim 1, wherein the scaffold contains at least one pharmacological active ingredient selected from the group consisting of antimicrobials, antivirals, antibiotics, growth factors, blood clotting modulators, growth factors, transforming growth factor (TGF-β3), bone morphogenic protein (BMP-2), PTHrP, osteoprotegrin (OPG), Indian Hedgehog, RANKL, and insulin-like growth factor (IgF1), and mixtures and composite layers thereof and further thereon the scaffold contains a biocompatible glue.

16. A method of making the tear resistant scaffold of claim 1, comprising the steps of:
introducing biodegradable materials to at least one of a substrate having pores and a substitute having-substantially no pores to a biocompatible tear resistant substrate in form of at lest one a of layer coating, and impregnation, solid, semi-solid, gel and liquid form to the substrate and optionally providing the construct with cells.

17. Use of the scaffold of claim 1 for repair of tissue defects selected from the group consisting of tendon tears, ligament defects and intervertebral disc defects.

18. The scaffold according to claim 1, wherein the cells are at least one of dispersed in and dispersed on a surface adjacent to at least one of the biodegradable material and the substrate.

19. The scaffold according to claim 1, wherein the cells are dispersed throughout at least one of the biodegradable material and the substrate.
